(19) United States
(12) Patent Application Publication
(10) Pub. No.: US 2019/0209449 A1
(43) Pub. Date: Jul. 11, 2019

(54) COLOR PROTECTION HAIR TREATMENT AGENT WITH EARTH ALKALINE SALTS OF DICARBOXYLIC ACIDS

(71) Applicant: Henkel AG & Co. KGaA, Duesseldorf (DE)

(72) Inventors: GEORG KNUEBEL, Duesseldorf (DE); THOMAS FOERSTER, Duesseldorf (DE)

(73) Assignee: Henkel AG & Co. KGaA, Duesseldorf (DE)

(21) Appl. No.: 16/325,682

(22) PCT Filed: Jun. 14, 2017
(86) PCT No.: PCT/EP2017/064495

§ 371 Date: Feb. 14, 2019

(30) Foreign Application Priority Data
Aug. 16, 2016 (DE) 10 2016 215 200.9

(51) Int. Cl. A61K 8/362; A61K 8/46; A61Q 5/00

(52) U.S. Cl. CPC A61K 8/362; A61K 8/463; A61K 2800/596; A61K 2800/884; A61K 2800/4324; A61Q 5/004

(57) ABSTRACT

The present disclosure relates to hair treatment agents for reducing and/or preventing the bleeding and/or fading of artificially created hair colours, which agents comprise an aqueous cosmetic carrier, and

(a) have a pH value ranging from about 3.5 to about 6.0, and
(b) contain at least one alkaline earth salt of a dicarboxylic acid having from about 2 to about 8 C atoms.

Further subjects of the present disclosure are a method and a multi-component packaging unit in which the aforesaid hair treatment agents are used.

A further subject of the present disclosure is the use of a corresponding hair treatment agent for protecting the colour of dyed hair.

### CROSS-REFERENCE TO RELATED APPLICATION

This application is a U.S. National-Stage entry under 35 U.S.C. § 371 based on International Application No. PCT/EP2017/064495, filed Jun. 14, 2017, which was published under PCT Article 21(2) and which claims priority to German Application No. 10 2016 215 200.9, filed Aug. 16, 2016, which are all hereby incorporated in their entirety by reference.

### TECHNICAL FIELD

The present disclosure lies in the field of cosmetics. The subject of the present disclosure is constituted by hair treatment agents for reducing and/or preventing the bleeding and/or fading of artificially created hair colours, which agents are based on an aqueous cosmetic carrier, have a pH value ranging from about 3.5 to about 6.0, and contain at least one alkaline earth salt of a dicarboxylic acid having from about 2 to about 8 C atoms.

### BACKGROUND

A further subject of the present disclosure is a method for reducing and/or preventing the bleeding and/or fading of artificially created hair colours, in which the hair is firstly coloured using a colourant and is then treated with a colour protector according to the first subject of the present disclosure.

A further subject of the present disclosure is a multi-component packaging unit (kit-of-parts) which comprises, packaged separately from one another in two containers, the agents (A) and (B), wherein the agent (A) is an agent for colouring hair and the agent (B) is a colour protector according to the first subject of the present disclosure.

Lastly, a further subject of the present disclosure is the use of an agent according to the first subject of the present disclosure for protecting the colour of dyed hair, or the use of the agent to reduce and/or prevent bleeding and/or fading of artificially created hair colours.

Human hair is nowadays treated in many different ways with hair-cosmetic preparations. These include, for example, the cleaning of hair with shampoos, the nourishment and regeneration with rinse-out treatments and leave-in conditioners, and the bleaching, dyeing and shaping of hair with colourants, toners, waving products and styling preparations. Agents for changing the colour of hair or for toning play a prominent role here.

In the field of hair colouring, there are in principle two types of hair colourants that are of significance: For permanent, intense colourations with corresponding fastness properties, what are known as oxidation dyes are used. Such colourants usually contain oxidation dye precursors, or what are known as developer components and coupler components. The developer components form the actual colouring dyes under the influence of oxidants or atmospheric oxygen, either on their own or with coupling to one or more coupler components. The oxidation dyes are exemplified by outstanding, long-lasting colour results. In many cases substantive dyes are also used additionally for toning.

For temporary colouring, usually dyes or toners that contain what are known as substantive dyes as colouring component are used. These are colourant molecules that are taken up directly on the hair and do not require an oxidative process in order to form the colour. These colourings are generally much more sensitive to shampoos than oxidative colourings, and therefore a change in tone or even a visible “decolouration” occurs a lot more quickly, which in many cases is undesirable.

After the colouring process, the hair is exposed over a long period of time to a wide range of environmental stresses. These range from daily wear and tear on the hair, for example caused by sunlight and hair washing, through stresses caused by basic hairdressing, as far as chemical influences if the consumer subjects their hair to a subsequent hair colouring or shaping process. The ambient influences not only have an effect on the hair structure itself, but also on the dyes remaining in the hair after the colouring process. The dyes can discolour as a result of exposure to the sun, or can be washed out from the hair by sweat or shampooing. The separation of the dyes from the hair fibres caused by shampoos or washing water is also referred to as bleeding. A person skilled in the art also summarizes the capability of the dyes to resist all of these ambient influences by the term “fastness properties”. If the dyes formed during the course of the colour formation or used directly have different fastnesses (for example UV stability, perspiration fastness, etc.), there may be an identifiable and undesirable colour shift over time. Both the occurrence of colour shifts and in particular the fading and bleeding of the colourings caused by washing are unwanted by the consumer.

Different colour protectors are already known in the prior art. These, however, are still worthy of improvement.

### BRIEF SUMMARY

Hair treatment agents for reducing and/or preventing the bleeding and/or fading of artificially created hair colours, methods for reducing and/or preventing the bleeding and/or fading of artificially created hair colours A hair treatment agent for reducing and/or preventing the bleeding and/or fading of artificially created hair colours, and multi-component packaging unit for the treatment of keratin fibres are provided herein. In an embodiment, a hair treatment agent for reducing and/or preventing the bleeding and/or fading of artificially created hair colours is provided, which agent includes an aqueous cosmetic carrier, and (a) has a pH value ranging from about 3.5 to about 6.0, and (b) includes at least one alkaline earth salt of a dicarboxylic acid having from about 2 to about 8 C atoms.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure was therefore to provide hair treatment agents and methods by employing which the bleeding and fading of artificially created hair colours can be reduced or, where possible, even avoided entirely.

It has now surprisingly been found that artificially dyed hair can be protected optimally against fading and bleeding if it is treated with an aqueous agent which has a pH value of from about 3.5 to about 6.0 and contains at least one alkaline earth salt of a dicarboxylic acid having from about 2 to about 8 C atoms.

A first subject of the present disclosure is a hair treatment agent for reducing and/or preventing the bleeding and/or fading of artificially created hair colours, which agent comprises an aqueous cosmetic carrier, and

(a) has a pH value ranging from about 3.5 to about 6.0, and
(b) contains at least one alkaline earth salt of a dicarboxylic acid having from about 2 to about 8 C atoms.

Suitable hair treatment agents are preferably hair cleansers, such as shampoos, haircare agents such as leave-in hair conditioners, rinse-out treatments or haircare sprays, and hair styling products, such as hair gels, hairsprays or hair waxes. The hair treatment agent is very particularly preferably a shampoo.

A preferred subject of the present disclosure is a shampoo for reducing and/or preventing the bleeding and/or fading of artificially created hair colours, which shampoo comprises an aqueous cosmetic carrier, and

(a) has a pH value ranging from about 3.5 to about 6.0, and
(b) contains at least one alkaline earth salt of a dicarboxylic acid having from about 2 to about 8 C atoms.

Artificially created hair colours are understood to mean colourings that can be produced by applying a hair colourant to hair. Here, these hair colourants contain at least one hair dye known from the prior art, in particular at least one oxidation dye precursor and/or at least one substantive dye.

The bleeding of artificially created hair colours can be noticed if dyed hair is treated either with water or with a surfactant-containing solution—such as a shampoo or a hair conditioner—and is then washed. A bleeding of the colouring is then noticeable in a colouring of the washing water. The bleeding does not lead only to a loss in intensity of the dyed hair, but also to a dirtying of textiles in the event of contact with the dyed washing water.

The fading of the artificially created hair dyes is understood to mean a loss in the intensity of the dyed hair. The extent to which hair colouring fades can be determined for example by comparative colorimetric measurements before and after hair washing.

The hair treatment agent comprises an aqueous cosmetic carrier. The cosmetic carrier can be aqueous or aqueous-alcoholic, for example. Suitable carriers are, for example, creams, emulsions, gels or surfactant-containing foaming solutions.

Aqueous-alcoholic solutions are understood to mean, in the sense of the present disclosure, in particular aqueous solutions containing from about 0.1 to about 70% by weight of a C₁-C₄ alcohol, in particular ethanol or isopropanol. The agents as contemplated herein can additionally contain further organic solvents, such as methoxybutanol, benzyl alcohol, ethyldiglycol or 1,2-propylene glycol. All water-soluble organic solvents are preferred here.

### pH Value

The hair treatment agent as contemplated herein has (a) a pH value ranging from about 3.5 to about 6.0. During the course of the works leading to this present disclosure it was found that the pH value when protecting the colour of dyed hair is a key influencing variable.

It was observed that the loss of colour intensity of dyed hair was minimal if the hair was treated with an agent set to a pH value ranging from about 3.6 to about 4.9, preferably from about 3.8 to about 4.8, more preferably from about 4.0 to about 4.7, and very particularly preferably from about 4.2 to about 4.6.

In the aforesaid preferred and particularly preferred pH value ranges, the bleeding of dyed hair fibres could also be suppressed to the best-possible extent.

In a particularly preferred embodiment a hair treatment agent as contemplated herein (a) has a pH value ranging from about 3.6 to about 4.9, preferably from about 3.8 to about 4.8, more preferably from about 4.0 to about 4.7, and very particularly preferably from about 4.2 to about 4.6.

The pH value can be measured for example using a glass electrode which is usually commercially available in the form of a combination electrode. The glass electrodes are usually calibrated using calibration solutions of known pH value prior to the measurement of the pH value. The pH values in the sense of the present disclosure are understood to be pH values that were measured at a temperature of 22° C.

Various acidifiers can be used in order to set the pH value that is essential to the present disclosure or preferred, Acidifiers that are preferred as contemplated herein are, for example, citric acid, lactic acid, acetic acid or also diluted mineral acids.

Although the hair treatment agents as contemplated herein are set to pH values in the acidic range, it may still be necessary also to use alkalising agents in small amounts in order precision adjust the pH value.

Alkalising agents that are suitable as contemplated herein are selected from the group formed from ammonia, alkanolamines, alkali metal hydroxides, alkali metal silicates, alkali metal phosphates and alkali metal hydrogen phosphates. Preferred inorganic alkalising agents are sodium hydroxide, potassium hydroxide, sodium silicate and sodium metasilicate. Organic alkalising agents that can be used as contemplated herein are preferably selected from monoethanolamine, 2-amino-2-methyl propanol and triethanolamine.

Alkaline Earth Salt of a Dicarboxylic Acid Having from about 2 to about 8 C Atoms.

As ingredient (b) essential to the present disclosure, the hair treatment agents contain at least one alkaline earth salt of a dicarboxylic acid having from about 2 to about 8 C atoms.

Dicarboxylic acids as contemplated herein are exemplified in that they have from about 2 to about 8 carbon atoms and precisely two carboxylic acid groupings. The two carboxylic acid groupings are present in deprotonated form, and the two negative charges are neutralised by the doubly positively charged alkaline earth cations (in particular Mg²⁺ and Ca²⁺). The carboxylic acids can also comprise one or more further substituents, such as hydroxy group(s), oxo group(s) or amino group(s). Dicarboxylic acid salts as contemplated herein can be aliphatic or aromatic, saturated or unsaturated.

Particularly well-suited alkaline earth salts of dicarboxylic acids having from about 2 to about 8 C atoms are understood as contemplated herein to mean in particular the magnesium salts and the calcium salts of these acids.

In a particularly preferred embodiment a hair treatment agent as contemplated herein contains (b) at least one magnesium salt and/or a calcium salt of a dicarboxylic acid having from about 2 to about 8 C atoms.

The strongest colour protection could be observed if dyed hair was treated with agents containing at least one calcium salt of a dicarboxylic acid having from about 2 to about 8 C atoms.

In a particularly preferred embodiment a hair treatment agent as contemplated herein contains (b) at least one calcium salt of a dicarboxylic acid having from about 2 to about 8 C atoms.

Within the group of alkaline earth salts of C₂-C₈ dicarboxylic acids as contemplated herein, certain compounds have proven to be particularly advantageous. For this reason it is very particularly preferred if the agents as contemplated herein contain at least one salt which is selected from the group of the calcium salt of succinic acid, the magnesium salt of succinic acid, the calcium salt of malonic acid, the magnesium salt of malonic acid, the calcium salt of maleic acid, the magnesium salt of maleic acid, the calcium salt of fumaric acid, the magnesium salt of fumaric acid, the calcium salt of glutaminic acid, the magnesium salt of glutaminic acid, the calcium salt of aspartic acid, the magnesium salt of aspartic acid, the calcium salt of oxalic acid, the magnesium salt of oxalic acid, the calcium salt of glutaric acid, the magnesium salt of glutaric acid, the calcium salt of adipic acid, the magnesium salt of adipic acid, the calcium salt of pimelic acid, the magnesium salt of pimelic acid, the calcium salt of suberic acid, the magnesium salt of suberic acid, the calcium salt of tartronic acid, the magnesium salt of tartronic acid, the calcium salt of tartaric acid, the magnesium salt of tartaric acid, the calcium salt of malic acid, the magnesium salt of malic acid, the calcium salt of 2-oxoglutaric acid, the magnesium salt of 2-oxoglutaric acid, the calcium salt of oxosuccinic acid and the magnesium salt of oxosuccinic acid.

Succinic acid is also known by the alternative name butanedioic acid. The calcium salt of succinic acid is referred to as calcium succinate. The calcium salt of succinic acid has the structural formula Ca²⁺(⁻OOC—CH₂—CH₂—COO⁻) and the CAS no. 140-99-8.

The magnesium salt of succinic acid is referred to as magnesium succinate. The magnesium salt of succinic acid has the structural formula Mg²⁺(⁻OOC—CH₂—CH₂—COO⁻).

Malonic acid is propanedioic acid. The calcium salt of malonic acid has the structural formula Ca²⁺(⁻OOC—CH₂—COO⁻). The magnesium salt of malonic acid has the structural formula Mg²⁺(⁻OOC—CH₂—COO⁻).

Maleic acid is also referred to as cis-butenedioic acid. The calcium salt of maleic acid has the structural formula (I). The magnesium salt of maleic acid has the structural formula (II).

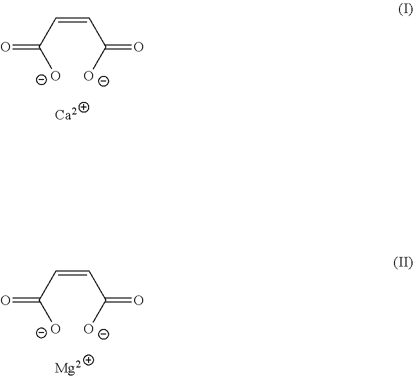 [SMILES: O=C([O-])/C=C\C(=O)[O-] ; O=C([O-])/C=C\C(=O)[O-]]

Fumaric acid also has the alternative name trans-butenedioic acid. The calcium salt of fumaric acid has the structural formula (III). The magnesium salt of fumaric acid has the structural formula (IV).

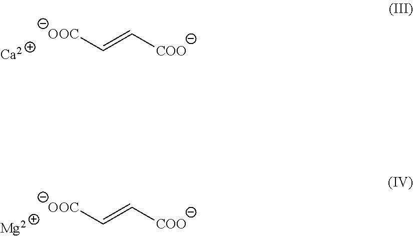

Glutaminic acid is the amino acid 2-aminopentanedioic acid, which occurs in the form of two enantiomers. The alkaline earth salts of (D)-glutaminic acid, (L)-glutaminic acid, and mixtures thereof are alkaline earth salts as contemplated herein. The calcium salt of glutaminic acid has the structural formula (V). The magnesium salt of glutaminic acid has the structural formula (VI).

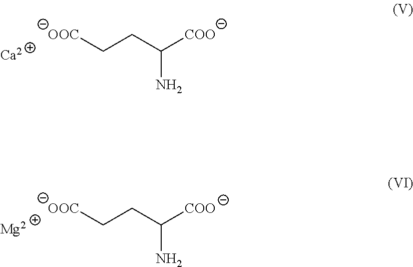

Aspartic acid is the amino acid 2-aminobutanedioic acid, which occurs in the form of two enantiomers. The alkaline earth salts of (D)-aspartic acid, (L)-aspartic acid, and mixtures thereof are alkaline earth salts as contemplated herein. The calcium salt of aspartic acid has the structural formula (VII). The magnesium salt of aspartic acid has the structural formula (VIII).

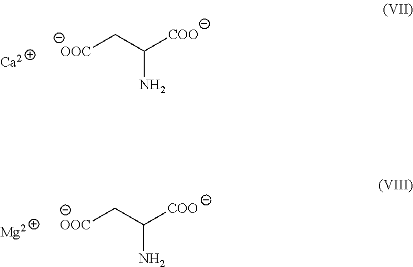

Oxalic acid is also referred to as ethanedioic acid. The calcium salt of oxalic acid has the alternative name calcium oxalate and has the structure formula Ca²⁺(⁻OOC—COO⁻). The magnesium salt of oxalic acid has the structural formula Mg²⁺(⁻OOC—COO⁻).

Glutaric acid is also referred to alternatively as pentanedioic acid. The calcium salt of glutaric acid has the structural formula Ca²⁺(⁻OOC—CH₂—CH₂—CH₂—COO⁻). The magnesium salt of glutaric acid has the structural formula Mg²⁺(⁻OOC—CH₂—CH₂—CH₂—COO⁻).

Adipic acid is also referred to as hexanedioic acid. The calcium salt of adipic acid has the structural formula Ca²⁺(⁻OOC—CH₂—CH₂—CH₂—CH₂—COO⁻). The magnesium salt of adipic acid has the structural formula Mg²⁺(⁻OOC—CH₂—CH₂—CH₂—CH₂—COO⁻).

An alternative name for pimelic acid is also heptanedioic acid. The calcium salt of pimelic acid has the structural formula Ca²⁺(⁻OOC—CH₂—CH₂—CH₂—CH₂—CH₂—COO⁻). The magnesium salt of pimelic acid has the structural formula Mg²⁺(⁻OOC—CH₂—CH₂—CH₂—CH₂—CH₂—COO⁻).

Suberic acid is octanedioic acid. The calcium salt of suberic acid has the structural formula Ca²⁺(⁻OOC—CH₂—CH₂—CH₂—CH₂—CH₂—CH₂—COO⁻). The magnesium salt of suberic acid has the structural formula Mg²⁺(⁻OOC—CH₂—CH₂—CH₂—CH₂—CH₂—CH₂—COO⁻).

Tartronic acid is alternatively also known as 2-hydroxypropanedioic acid. The calcium salt of tartronic acid has the structural formula Ca²⁺(⁻OOC—CH(OH)—COO⁻). The magnesium salt of tartronic acid has the structural formula Mg²⁺(⁻OOC—CH(OH)—COO⁻).

Tartaric acid, alternatively also referred to as 2,3-dihydroxysuccinic acid, has two optically active centres and occurs both in an L form, a D form and in a meso form. All of these forms and mixtures thereof can be used as contemplated herein. The calcium salt of tartaric acid is also referred to as calcium tartrate and has the structural formula

Ca²⁺(⁻OOC—CH(OH)—CH(OH)—COO⁻). The magnesium salt of tartaric acid is also referred to as magnesium tartrate and has the structural formula Mg²⁺(⁻OOC—CH(OH)—CH(OH)—COO⁻).

Malic acid, alternatively also referred to as 2-hydroxysuccinic acid, has an optically active centres and occurs both in an L form and a D form. Both of these forms and mixtures thereof can be used as contemplated herein. The calcium salt of malic acid has the structural formula Ca²⁺(⁻OOC—CH(OH)—CH₂—COO⁻). The magnesium salt of malic acid has the structural formula Mg²⁺(⁻OOC—CH(OH)—CH₂—COO⁻).

2-oxoglutaric acid is alternatively also known as 2-oxopentanedioic acid. The calcium salt of 2-oxoglutaric acid has the structural formula (⁻OOC—CH₂—CH₂—C(O)—COO⁻). The magnesium salt of oxoglutaric acid has the structural formula Mg²⁺(⁻OOC—CH₂—CH₂—C(O)—COO⁻).

An alternative name for oxosuccinic acid is also oxobutanedioic acid. The calcium salt of oxosuccinic acid has the structural formula Ca²⁺(⁻OOC—CH₂—C(O)—COO⁻). The magnesium salt of oxosuccinic acid has the structural formula Mg²⁺(⁻OOC—CH₂—C(O)—COO⁻).

In a preferred embodiment a hair treatment agent as contemplated herein (b) contains at least one alkaline earth salt of a dicarboxylic acid having from about 2 to about 8 C atoms which is selected from the group of the calcium salt of succinic acid, the magnesium salt of succinic acid, the calcium salt of malonic acid, the magnesium salt of malonic acid, the calcium salt of maleic acid, the magnesium salt of maleic acid, the calcium salt of fumaric acid, the magnesium salt of fumaric acid, the calcium salt of glutaminic acid, the magnesium salt of glutaminic acid, the calcium salt of aspartic acid, the magnesium salt of aspartic acid, the calcium salt of oxalic acid, the magnesium salt of oxalic acid, the calcium salt of glutaric acid, the magnesium salt of glutaric acid, the calcium salt of adipic acid, the magnesium salt of adipic acid, the calcium salt of pimelic acid, the magnesium salt of pimelic acid, the calcium salt of suberic acid, the magnesium salt of suberic acid, the calcium salt of tartronic acid, the magnesium salt of tartronic acid, the calcium salt of tartaric acid, the magnesium salt of tartaric acid, the calcium salt of malic acid, the magnesium salt of malic acid, the calcium salt of 2-oxoglutaric acid, the magnesium salt of 2-oxoglutaric acid, the calcium salt of oxosuccinic acid and the magnesium salt of oxosuccinic acid.

In a particularly preferred embodiment a hair treatment agent as contemplated herein (b) contains at least one alkaline earth salt of a dicarboxylic acid having from about 2 to about 8 C atoms which is selected from the group of the calcium salt of succinic acid, the magnesium salt of succinic acid, the calcium salt of malonic acid, the magnesium salt of malonic acid, the calcium salt of maleic acid, the magnesium salt of maleic acid, the calcium salt of fumaric acid, the magnesium salt of fumaric acid, the calcium salt of glutaminic acid, the magnesium salt of glutaminic acid, the calcium salt of aspartic acid, the magnesium salt of aspartic acid, the calcium salt of glutaric acid, the magnesium salt of glutaric acid, the calcium salt of adipic acid, the magnesium salt of adipic acid, the calcium salt of pimelic acid, the magnesium salt of pimelic acid, the calcium salt of suberic acid, the magnesium salt of suberic acid, the calcium salt of tartronic acid, the magnesium salt of tartronic acid, the calcium salt of tartaric acid, the magnesium salt of tartaric acid, the calcium salt of malic acid, the magnesium salt of malic acid, the calcium salt of 2-oxoglutaric acid, the magnesium salt of 2-oxoglutaric acid, the calcium salt of oxosuccinic acid and the magnesium salt of oxosuccinic acid.

In a very particularly preferred embodiment a hair treatment agent as contemplated herein contains (b) at least one alkaline earth salt of a dicarboxylic acid having from about 2 to about 8 C atoms which is selected from the group of the calcium salt of succinic acid, the calcium salt of malonic acid, the calcium salt of maleic acid, the calcium salt of fumaric acid, the calcium salt of glutaminic acid, the calcium salt of aspartic acid, the calcium salt of glutaric acid, the calcium salt of adipic acid, the calcium salt of pimelic acid, the calcium salt of suberic acid, the calcium salt of tartronic acid, the calcium salt of tartaric acid, the calcium salt of malic acid, the calcium salt of 2-oxoglutaric acid, the calcium salt of oxosuccinic acid and the magnesium salt of oxosuccinic acid.

Within the group of the aforementioned salts, the calcium salt of succinic acid (calcium succinate) is the most preferred.

In a very particularly preferred embodiment a hair treatment agent as contemplated herein contains (b) the calcium salt of succinic acid (calcium succinate).

It has been found that the best colour protection can be attained if the agent as contemplated herein contains the alkaline earth salts of the acid(s) in specific amount ranges. In this regard it has proven to be particularly advantageous to use one or more alkaline earth salts of dicarboxylic acids having from about 2 to about 8 C atoms in a total amount from about 0.1 to about 3.5% by weight, preferably from about 0.3 to about 2.8% by weight, more preferably from about 0.6 to about 2.4% by weight, and very particularly preferably from about 0.8 to about 1.6% by weight in the agent. All values in % by weight are understood here to relate to the total weight of the alkaline earth salts of the acids contained in the agent, which is set in relation to the total weight of the agent.

In a further very particularly preferred embodiment a hair treatment agent as contemplated herein contains—in relation to its total weight—(b) one or more alkaline earth salts of dicarboxylic acid having from about 2 to about 8 C atoms in a total amount from about 0.1 to about 3.5% by weight, preferably from about 0.3 to about 2.8% by weight, more preferably from about 0.6 to about 2.4% by weight, and very particularly preferably from about 0.8 to about 1.6% by weight.

As previously described, the use of calcium succinate in the hair treatment agent is very particularly preferred since the best effects could be attained with calcium succinate.

In an explicitly very particularly preferred embodiment a hair treatment agent as contemplated herein contains—in relation to its total weight—(b) from about 0.4 to about 2.6% by weight, preferably from about 0.7 to about 2.3% by weight, more preferably from about 0.9 to about 1.5% by weight, and very particularly preferably from about 1.2 to about 1.4% by weight calcium succinate.

### Anionic Surfactants

When measuring the washing fastness of dyed strands of hair it was noticed that an improved washing fastness and a reduced bleeding of the colourings could be obtained with the alkaline earth salts of C2-C8 dicarboxylic acids as compared to other salts (such as the alkali salts of these acids).

It has been found problematic, however, that the solubility of these alkaline earth salts is lower than the solubility of the corresponding alkali salts. Optimal results could therefore be attained in particular with best-possible solubilisation of the alkaline earth salts.

A sufficient solubility could be attained by choosing the optimal use amounts and pH values. In addition it has likewise been found, however, that the addition of at least one anionic surfactant to the agents dissolves the salts or disperses them finely to the best possible extent. For this reason it is very particularly preferred to additionally add at least one anionic surfactant to the agents as contemplated herein. In this regard the alkyl sulfates, alkyl ether sulfates, sulfosuccinates and ether carboxylic acids have proven to be particularly well suited.

In a further particularly preferred embodiment a hair treatment agent as contemplated herein contains at least one anionic surfactant from the group of alkyl sulfates, alkyl ether sulfates, ether carboxylic acids and sulfosuccinates.

Alkyl sulfate and/or alkyl ether sulfate salts have the general formula R—(OCH₂—CH₂)ₓ—OSO₃⁻M⁺, in which R is preferably a linear or branched, saturated or unsaturated alkyl group having from about 8 to about 30 C atoms, x represents the number 0 or from about 1 to about 12, and M means an alkali, ammonium or alkanolamine ion.

Particularly preferred anionic surfactants are straight-chain or branched alkyl sulfates of the aforementioned formula which contain an alkyl group having from about 8 to about 18, in particular having from about 10 to about 16 C atoms. In this case x stands for the number 10. Particularly preferred are the sodium, potassium and/or triethanol amine salts of linear or branched lauryl, tridecyl and/or myristyl sulfates.

Particularly preferred anionic surfactants are straight-chain or branched alkyl ether sulfates of the aforementioned formula which contain an alkyl group having from about 8 to about 18, in particular having from about 10 to about 16 C atoms, and from about 1 to about 6, in particular from about 2 to about 4 ethylene oxide units (i.e. x stands for an integer from about 1 to about 6, in particular for an integer from about 2 to about 4). Particularly preferred are the sodium, magnesium and/or triethanol amine salts of linear or branched lauryl, tridecyl and/or myristyl sulfates which have a degree of ethoxylation of from about 2 to about 4.

Preferred ethercarboxylic acids have the general formula R—O—(CH₂—CH₂O)ₓ—CH₂—COOM, in which R is a linear or branched, saturated or unsaturated alkyl group having from about 8 to about 30 C atoms and x=0 or from about 1 to about 16. M stands for a hydrogen atom or for an alkali, ammonium or alkanolamine ion.

If the hair treatment agent is formulated as a hair shampoo, it preferably contains at least one anionic surfactant in a preferred proportion by weight of from about 0.5 to about 20% by weight, more preferably from about 1 to about 15, and particularly preferably from about 2 to about 12% by weight, wherein the specified amounts relate to the total weight of the hair treatment agent.

The alkaline earth salts of C2-C8 dicarboxylic acids can solubilise or disperse particularly well by alkyl ether sulfates. It is therefore particularly preferred if the hair treatment agents contain at least one alkyl ether sulfate (for example in the form of the sodium salt), which—in relation to the total weight of the agent—is used in amounts of from about 5.5 to about 16.0% by weight, preferably from about 6.5 to about 14.0% by weight, more preferably from about 7.5 to about 12.0% by weight, and very particularly preferably from about 8.0 to about 10.0% by weight.

In a further particularly preferred embodiment a hair treatment agent as contemplated herein contains—in relation to its total weight—one or more anionic surfactants from the group of alkyl ether sulfates in a total amount from about 5.5 to about 16.0% by weight, preferably from about 6.5 to about 14.0% by weight, more preferably from about 7.5 to about 12.0% by weight, and very particularly preferably from about 8.0 to about 10.0% by weight.

Amphoteric and/or Zwitterionic Surfactants

It has also proven to be particularly preferred if the agents as contemplated herein contain at least one amphoteric and/or zwitterionic surfactant.

Surface-active compounds which carry at least one quaternary ammonium group and at least one —COO⁽⁻⁾ or —SO₃⁽⁻⁾ group in the molecule are referred to as zwitterionic surfactants. Particularly suitable zwitterionic surfactants are what are known as the betaines, such as N-alkyl-N,N-dimethylammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing in each case from about 8 to about 18 carbon atoms in the alkyl or acyl group and cocoacylaminoethyl hydroxyethyl carboxymethyl glycinate. A preferred zwitterionic surfactant is the fatty acid amide derivative known under the INCI name Cocamidopropyl Betaine.

Particularly well compatible with the alkaline earth salts of C₂-C₈ dicarboxylic acids are the betaines, in particular the N-alkyl-N,N-dimethylammonium glycinates and/or the N-acylaminopropyl-N,N-dimethyl ammonium glycinates.

Amphoteric surfactants are understood to be surface-active compounds which, apart from a C₈-C₂₄ alkyl or acyl group, also carry at least one free amino group and at least one —COOH— or —SO₃H group in the molecule and are capable of forming inner salts. Examples of suitable amphoteric surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkyl aminopropionic acids, and alkyl amino acetic acids each having approximately 8 to about 24 C atoms in the alkyl group. Typical examples of amphoteric and zwitterionic surfactants are alkyl betaines, alkyl amido betaines, amino propionates, amino glycinates, imidazolinium betaines and sulfobetaines. Particularly preferred amphoteric surfactants are N-coco-alkylaminopropionate, coco-acylaminoethylaminopropionate, and C₁₂-C₁₈ acyl sarcosine.

Particularly well compatible with the alkaline earth salts of C₂-C₈ dicarboxylic acids are 2-alkyl-3-carboxymethyl-3-hydroxyethyl-imidazolines, N-alkylglycines, N-alkylpropionic acid, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, alkylaminoacetic acids, N-alkylamphodiacetates and/or N-alkylamphodipropionates.

In a further particularly preferred embodiment a hair treatment agent as contemplated herein contains at least one amphoteric and/or zwitterionic surfactant from the group of betaines, 2-alkyl-3-carboxymethyl-3-hydroxyethyl-imidazolines, N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, alkylaminoacetic acids, N-alkylamphodiacetates and/or N-alkylamphodipropionates.

The amphoteric and/or zwitterionic surfactants are preferably used in the agent as contemplated herein in specific amounts which—in relation to the total weight of the agent—range from about 1.5 to about 6.5% by weight, preferably from about 2.5 to about 5.5% by weight, and particularly preferably from about 3.5 to about 4.5% by weight.

In a further preferred embodiment a hair treatment agent as contemplated herein contains—in relation to the total weight of the agent—one or more zwitterionic and/or amphoteric surfactants in a total amount from about 1.5 to about 6.5% by weight, preferably from about 2.5 to about 5.5% by weight, and particularly preferably from about 3.5 to about 4.5% by weight.

Explicitly very particularly preferred is the use of betaines, in particular of cocoamidopropyl betaine.

In a very particularly preferred embodiment a hair treatment agent as contemplated herein contains—in relation to its total weight—one or more zwitterionic surfactants from the group of betaines in a total amount from about 1.5 to about 6.5% by weight, preferably from about 2.5 to about 5.5% by weight, and particularly preferably from about 3.5 to about 4.5% by weight.

In a very particularly preferred embodiment a hair treatment agent as contemplated herein contains—in relation to its total weight—from about 1.5 to about 6.5% by weight, preferably from about 2.5 to about 5.5% by weight, and particularly preferably from about 3.5 to about 4.5% by weight cocoamidopropyl betaine.

### Further Ingredients

The hair treatment agents as contemplated herein can additionally contain further active substances, auxiliaries and additives, such as cationic surfactants, nonionic surfactants, anionic surfactants, nonionic and/or cationic polymers, structuring agents such as glucose, perfume oils, fibre structure-improving active substances, in particular mono-, di- and oligosaccharides, such as glucose, galactose, fructose, fruit sugars and lactose; colourants for colouring the agent; anti-dandruff active substances, such as piroctone, olamine, zinc omadine and climbazole; amino acids and oligopeptides; animal-based and/or plant-based protein hydrolysates, and those in the form of their fatty acid condensation products or optionally anionically or cationically modified derivatives; vegetable oils; light stabilisers and UV blockers; active substances such as panthenol, pantothenic acid, pantolactone, allantoin, pyrrolidinone carboxylic acids and colour-changing salts thereof and bisabolol; polyphenols, in particular hydroxycinnamic acids, 6,7-dihydroxycoumarin, hydroxybenzoic acids, catechins, tannins, leukoanthocyanidins, anthocyanidins, flavanones, flavones and flavonols; vitamins, pro-vitamins and vitamin precursors; plant extracts; swelling and penetrating substances such as glycerol, propylene glycol monoethyl ether, carbonates, hydrogen carbonates, guanidines, ureas and primary, secondary and tertiary phosphates; turbidity agents such as latex, styrene/PVP and styrene/acrylamide copolymers; pearlescent agents such as ethylene glycol mono- and distearate and PEG-3 distearate; pigments as well as propellants such as propane-butane mixtures, N₂O, dimethyl ether, CO₂ and air.

The additional active substances and auxiliaries are used in the agents as contemplated herein preferably in amounts of, in each case, from about 0.0001 to about 10% by weight, in particular from about 0.0005 to about 5% by weight, in relation to the total weight of the agent (A) or the oxidant preparation (B).

Method for Reducing and/or Preventing the Bleeding and/or Fading of Artificially Created Hair Colours

One purpose of the previously described hair treatment agents forming the first subject of the present disclosure is methods for reducing and/or preventing the bleeding and/or fading of artificially created hair colours. In other words, the agents as contemplated herein are used to protect the colour of artificially dyed hair. If the agents as contemplated herein are applied to hair that was dyed beforehand with oxidation dyes and/or substantive dyes, the fading, washing out, and bleeding of this hair are effectively minimised,

A second subject of the present disclosure is therefore a method for reducing and/or preventing the bleeding and/or fading of artificially created hair colours, which method comprises the following steps in the stated order

(I) applying a colourant to hair, wherein the colourant contains at least one oxidation dye precursor and/or at least one substantive dye

(II) leaving the colourant to act on the hair for a period of time from about 5 minutes to about 45 minutes

(III) rinsing out the colourant from the hair

(IV) applying a colour protector to the hair, wherein the colour protector is a hair treatment agent as described in detail in the description of the first subject of the present disclosure,

(V) leaving the colour protector to act for a period of time from about 5 minutes to about 45 minutes

In step (I) of the method as contemplated herein a colourant which contains at least one oxidation dye precursor and/or at least one substantive dye is firstly applied to the hair.

Oxidative colourings are created on the keratin fibres using oxidation dye precursors based on developer and coupler components. Oxidation dye precursors of the developer type are, for example, p-phenylenediamine, p-toluenediamine N,N-bis-(β-hydroxyethyl)-p-phenylenediamine, 4-N,N-bis-(β-hydroxyethyl)-amino-2-methylaniline, 2-(β-hydroxyethyl)-p-phenylenediamine, 2-(α,β-dihydroxyethyl)-p-phenylenediamine, 2-hydroxymethyl-p-phenylenediamine, bis-(2-hydroxy-5-aminophenyl)-methane, p-aminophenol, 4-amino-3-methylphenol, 2,4,5,6-tetraaminopyrimidine, 4-hydroxy-2,5,6-triaminopyrimidine, 2-hydroxy-4,5,6-triaminopyrimidine and/or 4,5-diamino-1-(β-hydroxyethyl)-pyrazole.

For example, m-phenylenediamine derivatives, naphthols, resorcinol and resorcinol derivatives, pyrazolones and m-aminophenol derivatives are used as oxidation dye precursors of the coupler type. The following are particularly suitable as coupler substances 1-naphthol, 1,5-, 2,7- and 1,7-dihydroxynaphthalene, 5-amino-2-methylphenol, m-aminophenol, resorcinol, resorcinol monomethyl ether, m-phenylenediamine, 1-phenyl-3-methyl-5-pyrazolone, 2,4-dichloro-3-aminophenol, 1,3-bis-(2′,4′-diaminophenoxy) propane, 2-chloro-resorcinol, 4-chloro-resorcinol, 2-chloro-6-methyl-3-aminophenol, 2-amino-3-hydroxypyridine, 2-methylresorcinol, 5-methylresorcinol and 2-methyl-4-chloro-5-aminophenol, 1-naphthol, 1,5-dihydroxynaphthalene, 1,7-dihydroxynaphthalene, 1,5-dihydroxynaphthalene, 3-aminophenol, 5-amino-2-methylphenol, 2-amino-3-hydroxypyridine, resorcinol, 4-chlororesorcinol, 2-chloro-6-methyl-3-aminophenol, 2-methylresorcinol, 5-methylresorcinol, 2,5-dimethylresorcinol and 2,6-dihydroxy-3,4-dimethylpyridine.

The colourants used in step (I) of the method can also contain one or more substantive dyes. In particular, nitrophenylenediamines, nitroaminophenols, azo dyes, anthraquinones or indophenols are potential substantive dyes. Preferred substantive dyes are the compounds known under the following international names or trade names: HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 12, Disperse Blue 3, Acid Blue 7, Acid Green 50, HC Violet 1, Disperse Violet 1, Disperse Violet 4, Acid Violet 43, Disperse Black 9, Acid Black 1, and Acid Black 52 and 1,4-diamino-2-nitrobenzene, 2-amino-4-nitrophenol, 1,4-bis-(β-hydroxyethyl)-amino-2-nitrobenzene, 3-nitro-4-(β-hydroxyethyl)-aminophenol, 2-(2′-hydroxyethyl)amino-4,6-dinitrophenol, 1-(2′-hydroxyethyl)-amino-5-chloro-2-nitrobenzene, 4-amino-3-nitrophenol, 1-(2′-ureidoethyl)amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid and 2-chloro-6-ethylamino-1-hydroxy-4-nitrobenzene.

Furthermore, the substrates to be decolourised can also be dyed using natural substantive dyes occurring in nature, as are contained for example in henna red, henna neutral, henna black, chamomile blossom, sandalwood, black tea, cascara bark, sage, logwood, madder root, catechu, sedr, and alkanet root.

A very particularly preferred method is exemplified by the step of

(I) applying an oxidative colourant to hair, wherein the oxidative colourant contains at least one oxidation dye precursor.

In an oxidative colourant the colourings are created under the influence of an oxidant (in particular hydrogen peroxide) from the oxidation dye precursors. The oxidative colourant applied in step (I) of the method is therefore a ready-to-use colourant which, besides the oxidation dye precursor(s), also contains at least one oxidant (in particular hydrogen peroxide).

A very particularly preferred method is therefore exemplified by the step of (I) applying an oxidative colourant to hair, wherein the oxidative colourant contains at least one oxidation dye precursor of the developer type, at least one oxidation dye precursor of the coupler type, and hydrogen peroxide.

The colourants applied to the hair in step (I) are then left to act in step (II) for a period of time of from about 5 to about 45 minutes and are then rinsed out in step (III).

Steps (I), (II) and (III) of the method are parts of the dyeing method and are therefore performed in direct chronological succession, i.e. within a period of time of a few hours.

Once the colourant has been rinsed out (step III), the hair can be dried optionally. It is also possible, however, to apply the colour protector (step (IV)) to hair which is still wet. The colour protector used in step (IV) is an agent according to the first subject of the present disclosure.

A period of time from a few hours to a number of days may lay between the execution of steps (III) and (IV).

Steps (III) and (IV) are preferably performed within a period of time from about 1 to about 72 hours, preferably from about 1 to about 48 hours, very particularly preferably from about 1 to about 6 hours.

Very particularly preferred is a method for reducing and/or preventing the bleeding and/or fading of artificially created hair colours, said method comprising the following steps in the stated order

(I) applying an oxidative colourant to hair, wherein the oxidative colourant contains at least one oxidation dye precursor of the developer type, at least one oxidation dye precursor of the coupler type, and hydrogen peroxide,

(II) leaving the dye to act on the hair for a period time from about 5 minutes to about 45 minutes,

(III) rinsing out the dye from the hair,

(IV) applying a colour protector to the hair, wherein the colour protector is a hair treatment agent as described in detail in the description of the first subject of the present disclosure,

(V) leaving the colour protector to act for a period of time from about 5 minutes to about 45 minutes,

wherein steps (III) and (IV) are performed within a period of time from about 1 to about 72 hours, preferably from about 1 to about 24 hours, very particularly preferably from about 1 to about 6 hours.

Once the colour protector has been applied, it is left to act in step (V) for a period of time from about 5 minutes to about 45 minutes.

The colour protector, i.e. the hair treatment agent as contemplated herein and forming the first subject of the present disclosure can be conceived as a “rinse-off” product or as a “leave-on” product.

As already described previously, however, it is very particularly preferred if the agent set to the appropriate pH values, in addition to water and the alkaline earth salts of C₂-C₈ dicarboxylic acids, additionally also contains the previously disclosed anionic and/or amphoteric/zwitterionic surfactants. If the agent contains these surfactants in higher amounts, formulation as a “rinse-off” product is preferred.

In a further very particularly preferred embodiment a method is exemplified in that step (V) is followed by a step of

(VI) rinsing out the colour protector from the hair.

Very particularly preferred is a method for reducing and/or preventing the bleeding and/or fading of artificially created hair colours, said method comprising the following steps in the stated order

(I) applying an oxidative colourant to hair, wherein the oxidative colourant contains at least one oxidation dye precursor of the developer type, at least one oxidation dye precursor of the coupler type, and hydrogen peroxide,

(II) leaving the dye to act on the hair for a period time from about 5 minutes to about 45 minutes

(III) rinsing out the dye from the hair

(IV) applying a colour protector to the hair, wherein the colour protector is a hair treatment agent as described in detail in the description of the first subject of the present disclosure,

(V) leaving the colour protector to act for a period of time from about 5 minutes to about 45 minutes,

(VI) rinsing out the colour protector from the hair,

wherein steps (III) and (IV) are performed within a period of time from about 1 to about 72 hours, preferably from about 1 to about 24 hours, very particularly preferably from about 1 to about 6 hours.

That said in respect of the hair treatment agent as contemplated herein applies mutatis mutandis to the preferred embodiments of the method as contemplated herein.

### Multi-Component Packaging Unit (Kit-of-Parts)

It is particularly convenient for the user, in order to carry out the previously described method, if all agents necessary for this purpose are provided to the user in the form of a multi-component packaging unit (kit-of-parts).

A third subject of the present disclosure is therefore a multi-component packaging unit (kit-of-parts) for the treatment of keratin fibres, in particular human hair, comprising, packaged separately from one another,
a container (I) containing a cosmetic agent (A) and
a container (II) containing a cosmetic agent (B), wherein
the agent (A) in container (I) is a colourant which contains at least one oxidation dye precursor and/or at least one substantive dye, and
the agent (B) in container (II) is a hair treatment agent as has been disclosed in detail in the description of the first subject of the present disclosure.

In the case of oxidative colourants, a ready-to-use colourant that was obtained shortly before use by mixing a colouring cream (containing the oxidation dye precursor(s)) and an oxidant preparation (containing hydrogen peroxide as oxidant) is usually applied to the hair.

In this regard, very particularly preferred is a multi-component packaging unit (kit-of-parts) for the treatment of keratin fibres, in particular human hair, comprising, packaged separately from one another,
a container (I) containing a cosmetic agent (A1) and
a container (II) containing a cosmetic agent (A2) and
a container (III) containing a cosmetic agent (B),
   wherein
the agent (A1) in container (I) is a colourant which contains at least one oxidation dye precursor and/or at least one substantive dye, and
the agent (A2) in the container (II) is an oxidant preparation containing hydrogen peroxide, and
the agent (B) in container (III) is a hair treatment agent as has been disclosed in detail in the description of the first subject of the present disclosure.

It is furthermore not ruled out for the multi-component packaging unit as contemplated herein to contain—besides the containers (I), (II) and possibly (III)—also a further container (IV). This container (IV) can contain, for example, a second portion of the colour protector or also another agent (for example a conditioner).

That said in respect of the hair treatment agent and method as contemplated herein applies mutatis mutandis to the preferred embodiments of the multi-component packaging unit as contemplated herein.

A further subject of the present disclosure is the use of the hair treatment agent forming the first subject of the present disclosure for protecting the colour of dyed hair.

A further subject of the present disclosure is the use of the hair treatment agent forming the first subject of the present disclosure to reduce and/or prevent the bleeding and/or fading of artificially created hair colours.

That said in respect of the hair treatment agent, method and kit-of-parts as contemplated herein applies mutatis mutandis to the preferred embodiments of the use as contemplated herein.

### Examples

### 1. Dyeing Strands of Hair

The following colourants were produced (all values in % by weight)

| | FC1 | FC2 |
|---|---|---|
| Cetearyl alcohol | 12.0 | 12.0 |
| Ammonium hydroxide | 4.7 | 4.7 |
| Glyceryl monostearate | 3.4 | 3.4 |
| Ceteareth-20 | 2.9 | 2.9 |
| 2-octyldodecanol | 2.0 | 2.0 |
| Sodium laureth sulfate (C12-C14, 2-3 EO) | 1.2 | 1.2 |
| Glycerol | 1.0 | 1.0 |
| Sodium cetearyl sulfate | 0.7 | 0.7 |
| Oleic acid | 0.4 | 0.4 |
| Sodium sulfite | 0.4 | 0.2 |
| Potassium stearate | 0.3 | 0.3 |
| Ethanolamine | 0.2 | 0.4 |
| EDTA, tetrasodium salt | 0.2 | 0.2 |
| Carbomer (polyacrylic acid, homopolymer) | 0.15 | 0.15 |
| Polyquatemium-39 | 0.15 | 0.15 |
| Potassium hydroxide | 0.05 | 0.05 |
| Ascorbic acid | 0.05 | 0.05 |
| Linoleamidopropyl PG-Dimonium Chloride | 0.03 | 0.03 |
| Phosphate | | |
| Titanium dioxide | 0.5 | 0.5 |
| 1-Hydroxyethyl 4,5-Diamino Pyrazole Sulfate | 1.90 | 0.94 |
| 4-amino-2-hydroxytoluene | 0.80 | — |
| Toluene-2,5-diamine sulfate | 0.35 | 0.04 |
| m-aminophenol | 0.30 | 0.06 |
| 1-naphthol | — | 0.33 |
| 6-methoxy-2-methylamino-3-aminopyridine HCl | — | 0.21 |
| 2-amino-4-[(2-hydroxyethyl)amino]-anisol, sulfate | — | 0.08 |
| Water | to 100 | to 100 |

The colouring creams FC1 and FC2 are each mixed in a ratio of 1:1 with a 6% aqueous hydrogen peroxide solution (OX). The ready-to-use colourant produced in this way was applied to damaged strands of hair (Kerling European natural hair 7/0, 1× ultra-bleached), left there for 30 minutes and then rinsed out with water. After drying, the dyed strands of hair were measured colorimetrically (measurement of the Lab values, Datacolor Spectraflash SF 450, illuminant: D65, program Datacolor Tools 2.0.1).

### 2. Colour-Protecting Shampoos

The following shampoos were produced (all values in % by weight)

| | Shampoo | Shampoo | Shampoo | Shampoo |
|---|---|---|---|---|
| | V1 | V2 | V3 | E1 |
| Sodium laureth sulfate (C12-C14, 2-3 EO) | 8.8 | 8.8 | 8.8 | 8.8 |
| Sodium hydroxide | 0.05 | 0.05 | 0.05 | 0.05 |
| Benzophenone-4 | 0.1 | 0.1 | 0.1 | 0.1 |
| Euperlan PK 3000 AM ⁽¹⁾ | 2.0 | 2.0 | 2.0 | 2.0 |
| Sodium benzoate | 0.5 | 0.5 | 0.5 | 0.5 |
| D-panthenol | 0.2 | 0.2 | 0.2 | 0.2 |
| Croquat WKP PE LQ ⁽²⁾ | 0.05 | 0.05 | 0.05 | 0.05 |
| Disodium cocoamphodiacetate | 2.0 | 2.0 | 2.0 | 2.0 |
| Coconut fatty acid monoethanolamide | 0.7 | 0.7 | 0.7 | 0.7 |
| PEG-7 glycerylcocoate | 0.85 | 0.85 | 0.85 | 0.85 |
| Hydrogenated Castor Oil | 0.25 | 0.25 | 0.25 | 0.25 |
| PEG-40 Hydrogenated Castor Oil | 0.3 | 0.3 | 0.3 | 0.3 |
| Apricot kernel oil | 0.1 | 0.1 | 0.1 | 0.1 |
| Polyquaternium-10 | 0.5 | 0.5 | 0.5 | 0.5 |
| Cocoamidopropyl betaine | 4.0 | 4.0 | 4.0 | 4.0 |
| Calcium succinate | — | — | — | 1.3 |
| Calcium lactate | — | — | 1.3 | — |
| Magnesium citrate (trimagnesium citrate) | — | 1.3 | — | — |
| Sodium lactate | 1.3 | — | — | — |
| Lactic acid | to pH 5.5 | to pH 4.5 | to pH 4.5 | to pH 4.5 |
| Water | to 100 | to 100 | to 100 | to 100 |

Euperlan PK 3000 AM ⁽¹⁾ = (AQUA (WATER), GLYCOL DISTEARATE, GLYCERIN, LAURETH-4, COCAMIDOPROPYL BETAINE, FORMIC ACID (BASF)
Croquat WKP PE LQ ⁽²⁾ = Cocodimonium Hydroxypropyl Hydrolyzed Keratin (Croda)

### 3. Measurement of the Colour Protection (Stir Test)

A 20% aqueous solution was produced from each of the shampoos V1, V2, V3 and E1. These solutions were each filled into a beaker. The coloured strands of hair were each immersed fully in a solution. Each beaker was then placed on a magnetic stirrer, an agitator was added, and stirring was performed at 300 rpm. Once a treatment period of 4 h had elapsed, the strands of hair were removed, rinsed out for 60 s using mains water (20° C.) and dried.

The colour distance ΔE* between the untreated strands and the strands treated in a defined way was determined from the obtained L*a*b* values in accordance with the following formula and the mean value from all four strands was formed:

ΔE*=\sqrt{(Lᵢ−L₀)²+(aᵢ−a₀)²+(bᵢ−b₀)²}

Lᵢ, aᵢ, bᵢ=values after treatment (stir test)
L₀, a₀, b₀=values before treatment (stir test)

For the assessments of the colour protection it is true that this is classified as being all the better, the lower are the corresponding ΔE* values.

| | Shampoo | Shampoo | Shampoo | Shampoo |
|---|---|---|---|---|
| | V1 | V2 | V3 | E1 |
| FC1 + OX | | | | |
| ΔE* values | 15.77 | 14.32 | 12.81 | 12.5 |
| FC2 + OX | | | | |
| ΔE* values | 15.7 | 12.6 | 12.4 | 12.2 |

### 4. Measurement of the Washing Fastness

In order to determine the resistance to washing, a 2% shampoo solution of each of the shampoos V1, V2, V3 and E1 was filled into an ultrasonic bath as far as the upper fill mark. The coloured strands of hair were then fully immersed and treated in the ultrasonic bath (11 min corresponds to 6 hair washes; the washing water was changed at each washing interval).

Three braids per tone and washing cycle were measured colorimetrically directly after the dyeing and after 24 hair washes, in each case at four different points (two at the front and two at the rear).

The colour distance ΔE* between the unwashed strands and the strands washed in a defined way was determined from the obtained L*a*b* values in accordance with the following formula and the mean value from all four strands was formed:

ΔE*=\sqrt{(Lᵢ−L₀)²+(aᵢ−a₀)²+(bᵢ−b₀)²}

Lᵢ, aᵢ, bᵢ=values after 24 hair washes
L₀, a₀, b₀=values after 0 hair washes

For the assessments of the washing fastness it is true that this is classified as being all the poorer, the higher are the corresponding ΔE* values.

| | Shampoo | Shampoo | Shampoo | Shampoo |
|---|---|---|---|---|
| | V1 | V2 | V3 | E1 |
| FC1 + OX, | | | | |
| 24 hair washes | | | | |
| ΔE* values | 5.3 | 5.7 | 5.4 | 5.1 |
| FC2 + OX, 24 | | | | |
| hair washes | | | | |
| ΔE* values | 5.4 | 4.9 | 4.7 | 4.3 |

### 5. Measurement of the Bleeding

Each shampoo V1, V2, V3 and E1 was applied to a coloured strand of hair in a liquor ratio of 1:5 (1 part shampoo, 5 parts hair). The strand of hair treated in this was placed in a watch glass filled with 50 ml water and was left there for 10 minutes. The colouring of the water was assessed visually under a daylight lamp (scoring system: 1=very weak bleeding, 6=very severe bleeding).

| | Shampoo | Shampoo | Shampoo | Shampoo |
|---|---|---|---|---|
| | V1 | V2 | V3 | E1 |
| FC1 + OX | | | | |
| Bleeding | 5 | 3 | 3 | 2 |
| FC2 + OX | | | | |
| Bleeding | 4 | 3 | 4 | 2 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A hair treatment agent for reducing and/or preventing the bleeding and/or fading of artificially created hair colours, which agent comprises an aqueous cosmetic carrier, and
(a) has a pH value ranging from about 3.5 to about 6.0, and
(b) comprises at least one alkaline earth salt of a dicarboxylic acid having from about 2 to about 8 C atoms.

2. The agent according to claim 1, wherein the agent has a pH value ranging from about 3.6 to about 4.9.

3. The agent according to claim 1, wherein the at least one alkaline earth salt of the dicarboxylic acid (b) comprises at least one calcium salt of a dicarboxylic acid having from about 2 to about 8 C atoms.

4. The agent according to claim 1, wherein the at least one alkaline earth salt of the dicarboxylic acid having from about 2 to about 8 C atoms is selected from the group of the calcium salt of succinic acid, the magnesium salt of succinic acid, the calcium salt of malonic acid, the magnesium salt of malonic acid, the calcium salt of maleic acid, the magnesium salt of maleic acid, the calcium salt of fumaric acid, the magnesium salt of fumaric acid, the calcium salt of glutaminic acid, the magnesium salt of glutaminic acid, the calcium salt of aspartic acid, the magnesium salt of aspartic acid, the calcium salt of oxalic acid, the magnesium salt of oxalic acid, the calcium salt of glutaric acid, the magnesium salt of glutaric acid, the calcium salt of adipic acid, the magnesium salt of adipic acid, the calcium salt of pimelic acid, the magnesium salt of pimelic acid, the calcium salt of suberic acid, the magnesium salt of suberic acid, the calcium salt of tartronic acid, the magnesium salt of tartronic acid, the calcium salt of tartaric acid, the magnesium salt of tartaric acid, the calcium salt of malic acid, the magnesium salt of malic acid, the calcium salt of 2-oxoglutaric acid, the magnesium salt of 2-oxoglutaric acid, the calcium salt of oxosuccinic acid and the magnesium salt of oxosuccinic acid.

5. The agent according to claim 1, comprising—in relation to its total weight—the (b) one or more alkaline earth salts of dicarboxylic acid having from about 2 to about 8 C atoms in a total amount from about 0.1 to about 3.5% by weight.

6. The agent according to claim 1, comprising—in relation to its total weight—from about 0.4 to about 2.6% by weight calcium succinate as (b) the at least one alkaline earth salt of the dicarboxylic acid.

7. The agent according to claim 1, comprising at least one anionic surfactant chosen from the group of alkyl sulfates, alkyl ether sulfates, ether carboxylic acids and sulfosuccinates.

8. The agent according to claim 1, comprising—in relation to its total weight—one or more anionic surfactants chosen from the group of alkyl ether sulfates in a total amount from about 5.5 to about 16.0% by weight.

9. The agent according to claim 1, comprising at least one amphoteric and/or zwitterionic surfactant chosen from the group of betaines, 2-alkyl-3-carboxymethyl-3-hydroxyethyl-imidazolines, N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, alkylaminoacetic acids, N-alkylamphodiacetates and/or N-alkylamphodipropionates.

10. The agent according to claim 1, comprising—in relation to its total weight—one or more zwitterionic surfactants chosen from the group of betaines in a total amount from about 1.5 to about 6.5% by weight.

11. A method for reducing and/or preventing the bleeding and/or fading of artificially created hair colours, said method comprising the following steps in the stated order
(I) applying a colourant to hair, wherein the colourant comprises at least one oxidation dye precursor and/or at least one substantive dye,
(II) leaving the colourant to act on the hair for a period of time from about 5 minutes to 45 about minutes,
(III) rinsing out the dye from the hair,
(IV) applying a colour protector to the hair, wherein the colour protector is a hair treatment agent according to claim 1,
(V) leaving the colour protector to act for a period of time from about 5 minutes to about 45 minutes.

12. The method according to claim 11, wherein the step of
(I) applying the colourant to hair comprises applying an oxidative colourant to hair, wherein the oxidative colourant comprises at least one oxidation dye precursor.

13. The agent according to claim 11, wherein step (V) is followed by a step of
(VI) rinsing out the colour protector from the hair.

14. A multi-component packaging unit for the treatment of keratin fibres comprising, packaged separately from one another,
a container (I) comprising a cosmetic agent (A) and
a container (II) comprising a cosmetic agent (B),
wherein
the agent (A) in container (I) is a colourant which comprises at least one oxidation dye precursor and/or at least one substantive dye, and
the agent (B) in container (II) is a hair treatment agent according to claim 1.

15. (canceled)

16. (canceled)

17. The agent according to claim 1, wherein the (b) one or more alkaline earth salts of dicarboxylic acid comprises an alkaline earth salt of succinic acid.

18. The agent according to claim 1, comprising—in relation to its total weight—alkaline earth salt of succinic acid as the (b) one or more alkaline earth salts of dicarboxylic acid in an amount of from about 1.2 to about 1.4% by weight.

19. The agent according to claim 1, wherein the (b) one or more alkaline earth salts of dicarboxylic acid comprises calcium succinate.

20. The agent according to claim 1, comprising—in relation to its total weight—calcium succinate as the (b) one or more alkaline earth salts of dicarboxylic acid in an amount of from about 1.2 to about 1.4% by weight.

21. The agent according to claim 1, comprising—in relation to its total weight—one or more anionic surfactants chosen from the group of alkyl ether sulfates in a total amount from about 8.0 to about 10.0% by weight.

22. The agent according to claim 1, comprising—in relation to its total weight:
one or more anionic surfactants chosen from the group of alkyl ether sulfates in a total amount from about 5.5 to about 16.0% by weight;
from about 1.2 to about 1.4% by weight calcium succinate; and
one or more zwitterionic surfactants chosen from the group of betaines in a total amount from about 1.5 to about 6.5% by weight;
wherein the agent has a pH value ranging from 4.2 to 4.6.
